(19) United States
(12) Patent Application Publication
(10) Pub. No.: US 2022/0340933 A1
(43) Pub. Date: Oct. 27, 2022

(54) NANOPARTICLE COMPOSITIONS FOR GENE THERAPY

(71) Applicant: University College Dublin, Dublin (IE)

(72) Inventors: Wenxin Wang, Co. Dublin (IE); Sigen A, Dublin (IE); Irene Lara-Saez, Valencia (ES); Qian Xu, Dublin (IE); Jonathan O'Keeffe Ahern, Co. Kildare (IE)

(21) Appl. No.: 17/763,441

(22) PCT Filed: Sep. 22, 2020
(86) PCT No.: PCT/EP2020/076449

§ 371 Date: Mar. 24, 2022

(30) Foreign Application Priority Data
Sep. 25, 2019 (EP) 19199697.4

(51) Int. Cl. C12N 15/88; C12N 15/90; A61K 9/51

(52) U.S. Cl. CPC C12N 15/88; C12N 15/90; A61K 9/5107; C12N 2310/20

(57) ABSTRACT

A nanoparticulate composition comprises a gene editing ribonucleoprotein system complexed within a cationic polymer. The cationic polymer may be a Poly-beta amino ester hyperbranched polymer, especially a 4-branching hyperbranched polymer. The gene editing ribonucleoprotein system may be a CRISPR-Cas9 gene editing system configured to excise a mutation or exon in a gene, replace a mutation in a gene, or produce a knock-down or knock-out of a gene, and in particular configured to excise exon 80 of the COL7A1 gene which codes for the collagen VII protein. Data shows that using

### FIELD OF THE INVENTION

The present invention relates to a nanoparticulate composition for gene therapy. Also contemplated are methods of treating skin genetic disorders such as Recessive dystrophic epidermolysis bullosa (RDEB).

### BACKGROUND TO THE INVENTION

Recessive dystrophic epidermolysis bullosa (RDEB) currently has no clinical therapy beyond palliative care and therefore a therapy to restore the structural integrity of skin by conferring type VII collagen expression to the patients' own cells is greatly required. Our research groups expertise lies in designing novel ways to introduce nucleic acids into cells and tissues. Current nucleic acid therapeutic approaches to restore type VII collagen expression suffer from concerns regarding safety and efficacy in delivering the treatment to cells and tissues. Genome editing is a way of making specific changes to the DNA of a cell and can be used to treat disorders like RDEB by repairing disease causing mutations. The previous genome editing technologies (ZFN's and TALEN's) have already been used for therapeutic approaches for RDEB. In the recent years a new safer and more versatile genomic editing technology (CRISPR) has garnered significant interest with its high therapeutic potential for patients with genetic diseases.

Development of a safe and efficient delivery systems is therefore crucial for the success of CRISPR genomic editing in clinics. Although the potential is huge, overcoming barriers to efficient delivery remains crucial for achieving safe and effective clinical success. Current methods to deliver CRISPR into cells are through; (1) a viral vector; (2) cell electroporation; or (3) polymer vehicles. Transient expression of therapeutic Cas9 and guide RNAs via non-viral delivery avoids an immune response caused by persistent expression of Cas9 and reduces off-target effects in vivo. A safe non-viral polymer delivery vector for a CRISPR system approach that can be used in a gentle manner to achieve correction of type VII collagen for RDEB patients would avoid substantial complication and invasive procedures associated with this debilitating disorder.

O'Keefe Ahern et al (Journal of Investigative Dermatology Vol. 138, No. 5. 19 May 2018, pages S141-S141) describes CRISPR/Cas9 based COL7A1 genome editing in recessive dystrophic epidermolysis bullosa (RDEB) via non-viral polymer delivery systems specifically a highly branched poly(beta-amino ester) polymer which binds electrostatically to negatively charged nucleic acids (plasmid DNA).

Zeng et al. (Nano Letters, Vol. 19, No. 1, 19 December 2018, pages 381-391) describes particles comprising DNA and poly(beta-amino ester) polymers and their use in fibroblast gene transfection (plasmid DNA).

Zeng et al. (ACS APPLIED MATERIALS & INTERFACES, Vol. 11, No. 34, 28 August 2019, pages 30661-30672) describes nanoparticles comprising minicircle COL7A1 DNA and branched poly(beta-amino) esters.

WO2019/104058 describes delivery of nucleic acid, including ribonucleoproteins, using core-shell structured nanoparticles with a poly (beta-amino ester) core enveloped by a phospholipid bilayer.

Kang et al. (Bioconjugate Chem 2017, 28, 957-967) describes non-viral genome editing that employs nanosized CRISPR complexes comprising PEI covalently bound to Cas9 protein, which is then complexed with a single-guide RNA molecule.

Chen et al. (ACS APPLIED MATERIALS & INTERFACES, 2018, 10, 18515-18523) describes polyplexes formed between nucleic (DNA, RNA or a Cas9/sgRNA ribonucleoprotein) and a cationic polymer poly(aspartic acid-(2-aminoethyl disulphide)-(4-imidazolecarboxylic acid))-poly(ethylene glycol).

Wang et al. (ACS APPLIED MATERIALS & INTERFACES, 2018, 10, 31915-31927) describes polyplexes formed between nucleic (DNA, RNA or a Cas9/sgRNA ribonucleoprotein) and a cationic copolymer, poly(N′N′-bis(acryloyl)cystamine-co-triethylenetetramine).

It is an object of the invention to overcome at least one of the above-referenced problems.

### SUMMARY OF THE INVENTION

The Applicant has discovered that hyperbranched poly (beta-amino) ester polymers are capable of efficiently condensing ribonucleoprotein complexes into nanoparticles to protect them from enzyme degradation and facilitate transport across the cell membrane in an efficient and cytocompatible manner. Nanoparticulate compositions of the invention comprising a functional CRISPR-Cas9 collagen VII exon 80 excision system have been shown to transfect keratinocytes with high transfection and correction efficiency and high cell viability (FIGS. 5 to 7). These compositions are capable of penetrating into the epidermal base layer trough of Recessive dystrophic epidermolysis bullosa (RDEB) blisters after 2 days of topical and subdermal injection in vivo (FIG. 9) excising the exon 80 in vivo and ex vivo (FIG. 10) and restoring type VII collagen in vivo after 7 days of topical application of the compositions (FIG. 11). Compared with a CRISPR-plasmid system, the nanoparticle compositions of the invention exhibit higher transfection efficiency (FIG. 12) and higher correction efficiency (8.2% to 43.2%-FIG. 13). The invention broadly relates to nanoparticulate compositions comprising hydrophobic cationic polymers and ribonucleoprotein complexes of CRISPR-Cas derivatives, their uses in gene therapy (especially skin genetic disorders), and in particular for the treatment of RDEB.

In a first aspect, the invention provides a nanoparticulate composition comprising a gene editing ribonucleoprotein system complexed within a cationic polymer for example a hyperbranched polymer (hereafter “nanoparticulate composition” or “ribopolyplex”).

In one embodiment, the hyperbranched polymer is a poly(beta amino ester) hyperbranched polymer.

In one embodiment, the hyperbranched polymer is a 3-branching or 4-branching hyperbranched polymer.

In one embodiment, the gene editing ribonucleoprotein system is a Cas9-gRNA ribonucleoprotein system, such as a CRISPR-Cas9 gene editing system, which is typically configured to induce deletion of a targeted genomic sequence including excision of a mutation or exon in a gene, replace a mutation in a gene, or produce a knock-down or knock-out of a gene. Other gene editing ribonucleoprotein systems that may be employed with the present invention include for example alternative CRISPR-Cas derivatives such as Cas12a, Cas14, CRISPR Base editors, zinc finger nuclease systems and TALEN systems.

In one preferred embodiment, the gene editing ribonucleoprotein system is a CRISPR-Cas9 gene editing system.

In one embodiment, the gene editing ribonucleoprotein system is configured for exon-excision. In one embodiment, the gene editing ribonucleoprotein system is a CRISPR-Cas9 gene editing system. In one embodiment, the gene editing ribonucleoprotein system is configured to excise exon 80 of the COL7A1 gene encoding for Collagen VII protein.

In one embodiment, the ribopolyplex of the invention has an average dimension of 50-500, 50-400, 50-300, 100-400, 100-300, 150-250, and ideally about 200 nm. Methods of measuring the average size of the nanoparticulate compositions are for example a dynamic light scattering system or transmission electron microscopy. To measure ribopolyplex size, and polydispersity index (PDI), which provides a measurement of nanoparticle uniformity in solution, a Malvern Zetasizer Nano ZS (Malvern Instrument) equipped with a scattering angle of 173° can be used. Ribopolyplex size measurements are performed in a clear plastic disposable cuvette. Ribopolyplexes were prepared by firstly assembling the ribonucleoprotein through the mixing of sgRNA and Cas9 nuclease at the desired ratio between 1.1-9.0:1. Following assembly of the ribonucleoprotein, ribopolyplexes were formed by mixing polymer and ribonucleoprotein at a volume/volume ratio of 1:1 and allowing to incubate for 15 min at room temperature. Following incubation ribopolyplexes were further diluted with 980 μl of molecular water and added into a clear plastic disposable cuvette for measurement at a temperature of 25° C.

In another aspect, the invention provides a composition comprising a first nanoparticulate composition according to the invention comprising a first Cas9-gRNA ribonucleoprotein system and a second nanoparticulate composition according to the invention comprising a second Cas9-gRNA ribonucleoprotein system and in which the gRNA of the first Cas9-gRNA ribonucleoprotein system is different to the gRNA of the second Cas9-gRNA ribonucleoprotein system. These compositions are useful in exon excision where the first and second gRNA molecules are configured to anneal at opposed flanks of the target exon to be excised.

The invention also provides a conjugate comprising a ribopolyplex according to the invention, and an additional molecule, for example (a) a targeting ligand configured to target the nanoparticulate composition to a specific target cell or tissue type or (b) an imaging label or dye. The additional molecule may be conjugated to the protein or nucleic acid element of the composition, and may be conjugated covalently, or associated in another manner, for example by electrostatic interaction.

The invention also provides a pharmaceutical composition comprising a ribopolyplex or a conjugate of the invention in combination with a suitable pharmaceutical excipient.

The invention also provides a method of making a nanoparticulate composition comprising the steps of:
providing a solution of gene editing ribonucleoprotein system in a buffer;
providing a solution of cationic polymer in a suitable non-aqueous solvent;
mixing the solutions such that there is an excess of mass of the cationic polymer over that of the gene editing ribonucleoprotein system in the mixture; and
typically resting the mixture to allow the nanoparticulate composition to form.

In one embodiment, the cationic polymer solution is prepared by dissolving the cationic polymer is a suitable solvent (for example a non-aqueous solvent such as DSMO), and then dilution the solution in an aqueous buffer.

In one embodiment, the cationic polymer is dissolved in the solvent at a concentration of 10-200 mg/ml, preferably 50-150 mg/ml, and ideally about 100 mg/ml.

In one embodiment, the solution after dilution with the buffer comprises 0.1 to 100 g of cationic polymer.

In one embodiment, the method comprises a step of assembly of the gene editing ribonucleoprotein system.

In one embodiment the step comprises mixing a sgRNA with a Cas9 nuclease at a molar ratio between 1.1-9.0:1 to provide gene editing ribonucleoprotein system typically containing 0,1 to 100 μg of ribonucleoprotein complex.

In one embodiment, the ribonucleoprotein complex is diluted in a volume of a given buffer such that the final ribonucleoprotein complex solution does not exceed that of 50% of the total desired application volume.

In one embodiment, the first and second solutions are mixed at a volumetric ratio of about 1-100:1-100, such that there is an excess of mass of the polymer over that of the gene editing ribonucleoprotein system.

In one embodiment, the buffer is configured to have a pH in the range of 3 to 10. In one embodiment, the buffer comprises a buffering salt in a concentration of 10-50, preferably 20-30 mM. In one embodiment, the buffer is a sodium acetate buffer.

In one embodiment, the amount of cationic polymer in the second solution is 1 to 100 times more than the ribonucleoprotein complex in terms of mass, and in which the first and second solutions are mixed at a volumetric ratio of about 1-100:1-100.

The invention also provides a ribopolyplex comprising a gene editing ribonucleoprotein system complexed within a cationic polymer, for use in a method of treatment of a genetic disease in an individual (typically characterised by a mutation in a gene of the individual), wherein the gene editing ribonucleoprotein system is in one embodiment configured to edit the gene. Editing may comprise non-homologous end joining (NHEJ) (i.e. for large or small genomic deletions or exon excision), knock down or knock out a gene, for homology direct repair (HDR), adding a DNA template to the ribopolyplex. In a preferred embodiment, the editing comprising deleting or replacing the mutation or a section of the gene including the mutation.

In one embodiment, the ribopolyplex is administered topically or by sub-dermal injection.

In one embodiment, the genetic disease is selected from a skin genetic disorder. Examples include Epidermolysis Bullosa (EB), Recessive dystrophic epidermolysis bullosa (RDEB), Epidermolytic Palmoplantar Keratoderma, Hailey-Hailey's disease, Darier's disease, Localized Autosomal Recessive Hypotrichosis. Additional skin diseases may include: alternative EB subtypes such as Simplex EB and Junctional EB, Epidermolytic Palmoplantar Keratoderma, Hailey-Hailey's disease, Darier's disease and, Localized Autosomal Recessive Hypotrichosis,

In one embodiment, the genetic disease is Recessive Dystrophic subtype of Epidermolysis Bullosa (RDEB), and wherein the gene editing ribonucleoprotein system is configured for collagen VII exon 80 excision. Preferably, the gene editing ribonucleoprotein system is a CRISPR-Cas9 gene editing system.

In another aspect, the invention provides a method of treating a skin genetic disease in a subject comprising a step of administering a nanoparticulate composition according to the invention to the skin of the individual by topical administration or sub-dermal injection, in which the nanoparticulate composition of the invention comprises a gene editing ribonucleoprotein system typically comprising a CRISPR nuclease complexed with a cationic polymer. Typically, the CRISPR nuclease protein is Cas9 or a Cas9 derivative.

In another aspect, the invention provides a method of genetically modifying a cell ex-vivo or in-vitro comprising a step of incubating the cell with a nanoparticulate composition according to the invention, whereby the gene-editing ribonucleoprotein system genetically modifies the cell. In one embodiment, the method comprises a step of isolating the cell from a subject, and then implanting the genetically modified cell into the subject. In one embodiment, the subject has a genetic disease characterised by a mutation in a gene in the cell, wherein the gene-editing ribonucleoprotein system is configured to correct the mutation or delete the mutation or all or part of an exon containing the mutation.

In another aspect, the invention provides a method of genetically modifying a sample of tissue ex-vivo or in-vitro comprising a step of incubating the tissue with a nanoparticulate composition according to the invention, whereby the gene-editing ribonucleoprotein system genetically modifies at least some of the cells of the tissue. In one embodiment, the method comprises a step of isolating the tissue from a subject, and then implanting the genetically modified tissue into the subject. In one embodiment, the subject has a genetic disease characterised by a mutation in a gene in a cell of the tissue, wherein the gene-editing ribonucleoprotein system is configured to correct the mutation or delete the mutation or all or part of an exon containing the mutation.

In another aspect, the invention provides a cell or tissue genetically modified in-vitro or ex-vivo according to a method of the invention.

The invention also provides a ribopolyplex comprising a gene editing ribonucleoprotein system complexed within a cationic polymer, for use in a method of treatment of an inflammatory disease in an individual characterised by over-expression of an inflammatory mediator, wherein the gene editing ribonucleoprotein system is configured to edit the genome of the individual to effect a reduction in expression of the inflammatory mediator.

Other aspects and preferred embodiments of the invention are defined and described in the other claims set out below.

### BRIEF DESCRIPTION OF THE FIGURES

FIG. 1: Scheme defining the concept of ribopolyplex.

FIG. 2: Route of action of the ribopolyplexes.

FIG. 3: Scheme of the gene editing strategies using ribopolyplexes.

FIG. 4: Formation of the ribopolyplexes for RDEB treatment.

FIG. 5: Collagen exon 80 excision: the ribonucleoprotein (RNP) complex produces a double strand break flanking exon 80 that is removed and repaired the DNA strand by non-homologous end joining (NHEJ) and restored the collagen VII production.

FIG. 6: Cell viability of RDEBK after 72 hours transfection with ribopolyplexes. Transfected RDEB keratinocytes (RDEBK) with ribopolyplexes for collagen VII exon 80 excision showed high viability, comparable to untreated ones.

FIG. 7: TracrRNA marker 72 hours post-transfection of RDEBK with ribopolyplexes, scale 100 um. Fluorescence microscope images show the fluorescent red marker (tracr) after 72 hours post-transfection of RDEBK with 2 ug RNP complex after washing with Hank's. The ribopolyplexes showed the same (with P3 polymer), higher (polymers P1 and P2) and more diffused (Y4 polymer) signal than HPAE (FIG. 7).

FIG. 8: Electrophoresis gel of PCR product of the transfected cells DNA. Confirmation of the correction efficiency of the ribopolyplexes by electrophoresis gel of PCR product of primary transfected keratinocytes DNA. The exon was excised obtaining a shorter band with higher efficiency of ribopolyplexes (Y4 and P2) than with HPAE.

FIG. 9: Fluorescent image of a blister in a RDEB human graft showing fluorescently labelled red tracrRNA. In vivo application of the ribopolyplexes using different polymers in RDEB human graft model showed the penetration of the ribopolyplexes into the epidermal basal layer trough the RDEB blisters after 2 days of topical and subdermal injection of the ribopolyplexes by fluorescently labelled red tracrRNA.

FIG. 10: Gel electrophoresis results from humanized RDEB skin grafts amplified DNA (PCR) treated in vivo and ex vivo with 4-Branched Polymers gene editing systems. After 12 days of a single topical application in vivo in an induced wound model in a RDEB human graft, a correction of over 8% was detected (FIG. 10 left hand side). The excised portion of the graft obtained to create the wound in the animal was immersed in the same ribopolyplex solution that was applied onto the animal. Sample was analysed after 6 days of immersion and exon excision correction achieved the 33.65% (FIG. 10 right side).

FIG. 11: Type VII collagen protein fluorescent detection by immunohistochemistry of human RDEB skin graft samples after 7 days topical transfection with 4-Branched Polymers gene editing systems. Human type VII collagen expression was restored after 2 dose treatment application with P2 Polymers (FIG. 11 right hand side) confirmed by comparison with positive and negative controls for type VII collagen (FIG. 11 left side). Antibodies for the 2 amino-terminal non-collagenous domains NC1 (red) and NC2 (green) were used to ensure the functionality of the corrected expression of type VII collagen. Involucrin detection ensures the human origin of the grafted skin (red).

FIG. 12: Transfection efficiency of 3-Branched Polymers gene editing systems: Plasmid compared with Ribonucleoprotein (RNP). Immortalised human RDEB keratinocytes containing a mutation exon 80 were transfected with a CRISPR plasmid/3-branched polymer complex and with a CRISPR-RNP/3-branched polymer ribopolyplex. Low transfection efficiency was achieved with the plasmid system (left figure) and high transfection efficiency achieved with the RNP system.

FIG. 13: Correction efficiency of 3-Branched Polymers gene editing systems: Plasmid compared with Ribonucleoprotein (RNP): FIG. 13 shows two different PCR amplicon sizes as a result of differing primer systems used. Using 3 branched polymers with CRISPR DNA plasmid achieved 8.2% correction efficiency however using the CRISPR RNP complex system the efficiency improved up to 43.2% with the same w/w ratio. The fluorescence microscope images in FIG. 12 along with the PCR results show that the RNP complex achieved higher transfection and correction efficiency.

FIG. 14: Transfection efficiency of gene editing Ribopolyplexes: 3-Branched Polymers v 4-Branched Polymers. Correction efficiencies achieved with Y4 Polymers and CRISPR RNP complex system reached 65.98% in pig primary keratinocytes, significantly higher than that achieved with 3-branched polymers in immortalized cells (FIG. 14 left side). Correction efficiency using Y4 polymers and was achieved even in RDEB human primary keratinocytes, a known difficult to transfect cell (FIG. 14 right side).

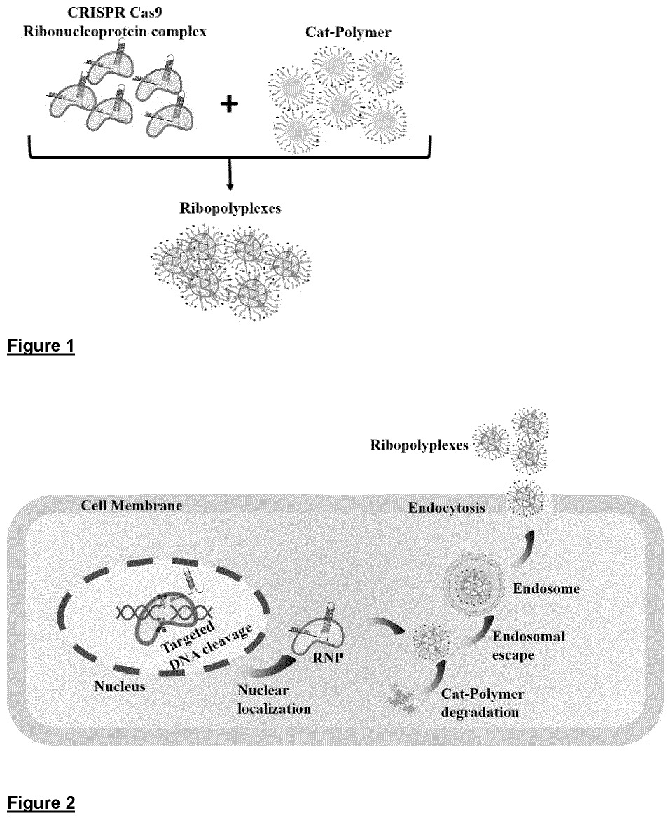

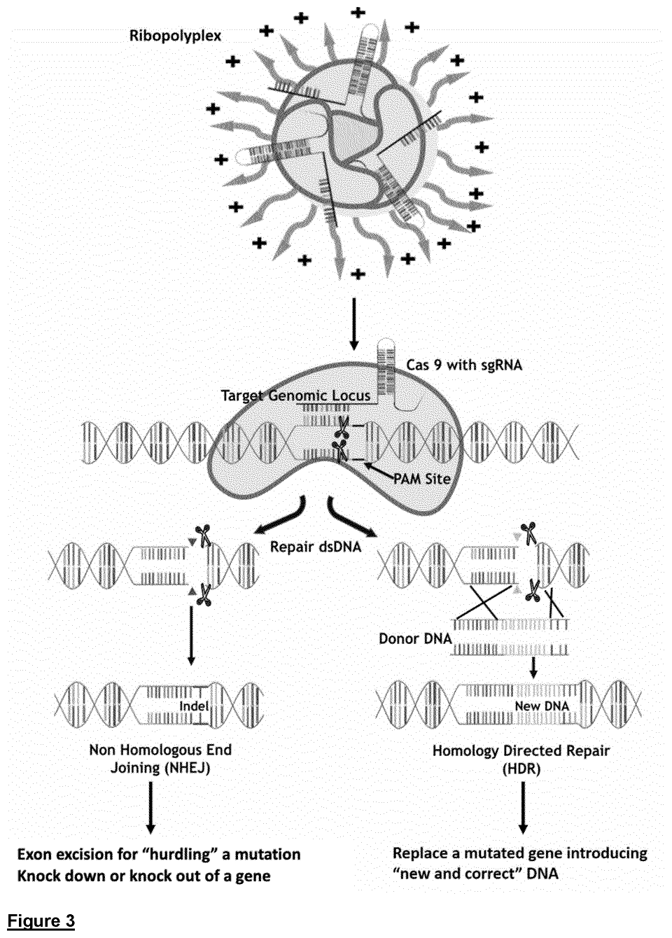

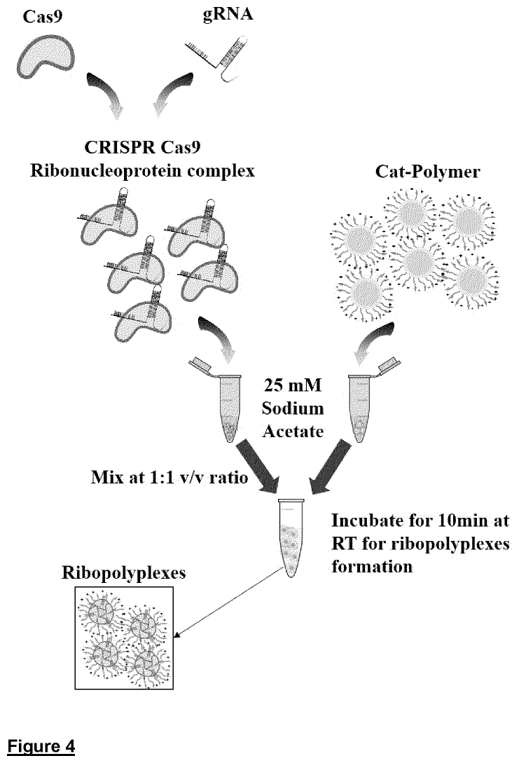

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:
Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, assess, kiang and zebra.

“Gene editing ribonucleoprotein system” or “gene editing RNP system” refers to a complex formed by a ribosomal protein bound to one or more sequences of nucleic acid that is capable of editing a gene in a mammal, for example by deleting or replacing a mutation in a gene or a segment of a gene (such as an exon), inserting an oligonucleotide into a gene (insertional mutagenesis), or modulating the expression of a gene (knock-down or knock-out mutation). The nucleic acid may be RNA in a format consisting of but no limited to, crRNA, tracRNA, sgRNA. Generally the nucleic acid is a sgRNA comprising crRNA and tracrRNA. The ribosomal protein may be a CRISPR nuclease protein, e.g. Cas9, Cas12a, Cas14 or a Cas variant, for example modified versions of nuclease dead (dCas9). The gene editing ribonucleoprotein system can be further complimented with the addition of nucleic acids in the form of DNA or RNA or a combination of both. Complimentary nucleic acids can be incorporated into the gene editing ribonucleoprotein system to induce gene augmentation, gene silencing, gene addition, gene knockdown, gene knockout, gene editing via homology directed repair. In some embodiments the nucleic acid may be employed in a format consisting of but not limited to RNA oligonucleotides, antisense oligonucleotides. DNA may be employed in a format consisting of but not limited to DNA oligonucleotides, antisense oligonucleotides, single-strand DNA donor oligo, plasmid DNA. The gene editing system may be a CRISPR-associated Cas system (Sander and Joung (2014) CRISPR-Cas systems for editing, regulating and targeting genomes Nature Biotechnology 32(4): 347-355)), a TALEN system (Boch J (February 2011); “TALEs of genome targeting”. Nature Biotechnology. 29 (2): 135-6. doi:10.1038/nbt.1767. PMID 21301438), a meganuclease system, or a zinc finger nuclease (ZFN) system (Carroll, D (2011) “Genome engineering with zinc-finger nucleases”. Genetics Society of America. 188 (4): 773 78doi:10.1534/genetics.111.131433. PMC 3176093. PMID 21828278). In one embodiment, the gene editing system is configured to perform insertational mutagenesis on a cell, for example OBLIGARE systems, and CRISPR-Cpf1 systems (Maresca et al. (2013) Obligate Ligation-Gated Recombination (ObLiGaRe): Custom-designed nuclease-mediated targeted integration through nonhomologous end joining Genome Res. 23: 539-546; see also WO2014/033644), Fagerlund et al. (2015) The Cpf1 CRISPR-Cas protein expands genome-editing tools Genome Biology 16: 251-253; Ledford (2015) Bacteria yield new gene cutter Smaller CRISPR enzyme should simplify genome editing Nature 526: 17). The gene editing ribonucleoprotein system of the invention may be employed in gene addition, gene replacement, gene knockdown and gene editing. Gene replacement is defined as the provision of a functional healthy copy of a gene to replace a dysfunctional mutant containing gene which has given rise to a disease. Gene addition is defined as the supplementation of therapeutic genes that target a specific aspect of a disease mechanism. Gene knockdown is defined as the process of inhibiting a target genes capability to synthesize a toxic/dysfunctional protein which gives rise to a disease. Gene editing is defined as the process whereby a target genes nucleotide sequence is altered resulting in either a loss of function/correction/manipulation of gene expression. Such gene editing systems consists of but are not limited to i) clustered, regularly interspaced, palindromic repeats (CRISPR)-associated (Cas) system; (ii) a transcription activator-like effector nuclease (TALEN) system; or (iii) a zinc finger nuclease (ZFN) system.

“Cationic polymer” refers to polymers with positive charges. E.g. LPAE, HPAE, LBPAE, poly-beta amino ester hyperbranched polymers, hyperbranched polymers, hyperbranched poly-beta amino ester polymers, and hyperbranched PEG polymers.

“Poly-beta amino ester hyperbranched polymer” refers to a cationic polymer formed by random polymerisation between branched monomers (for example monomers having three, four or more reactive sites that can react with acrylate or amine groups), diacrylate groups and first and second amine components to provide a highly branched poly(β-amino ester) (HPAE) having a 3-D structure and multiple end groups. The term includes 3-branching hyperbranched polymers and 4-branching hyperbranched polymers.

“3-branching hyperbranched polymer” refers to polymers formed by reacting a monomer with three reacting sites that can react with acrylate or amine groups (three-branching monomer) with a diacrylate and first and second amine components. In one embodiment, the polymer is formed using a oligomer combination approach, in which the diacrylate and first amine components are reacted together to form a first oligomer, the first oligomer and second amine component are reacted together to form a second oligomer, and the second oligomer and four branching monomer are reacted together to form the hyperbranched polymer of the invention. This oligomer combination approach is described in detail in Zeng et al (Nano. Lett. 2019 19, 381-391). In another embodiment, the four-branching monomer, diacrylate component, and first amine are reacted together in a Michael Addition reaction to form a first polymer, and the first polymer and second amine component (endcapping amine) are reacted together in a Michael Addition reaction to form the hyperbranched polymer of the invention. Examples of 3-branching hyperbranched polymers are described in US2017216455 and Zeng et al.

4-4-branching hyperbranched polymer may be made by reacting together:
(i) a four-branching monomer with four reaction sites that can react with acrylate or amine groups;
(ii) a diacrylate component, typically of formula (I)

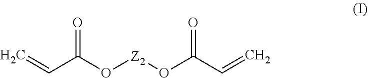

wherein Z2 is a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms;
wherein Z2 is unsubstituted or substituted with at least one of a halogen, a hydroxyl, an amino group, a sulfonyl group, a sulphonamide group, a thiol, a C1-C6 alkyl, a C1-C0 alkoxy, a C1-C6 ether, a C1-C6 thioether, a C1-C6 sulfone, a C1-C6 sulfoxide, a C1-C6 primary amide, a C1-C6 secondary amide, a halo C1-C5 alkyl, a carboxyl group, a cyano group, a nitro group, a nitroso group, —OC(0)NR′R′, —N(R′)C(0)NR′R, —N(R′)C(0)0-C1-C6 alkyl, C3-C6 cycloalkyl, C3-C6 heterocyclyl, C2-C5 heteroaryl and C6-C10 aryl; wherein each R′ is independently selected, from the group consisting of hydrogen and Ci-C6 alkyl;
(iii) a first amine component typically comprising 3 to 20 atoms,
   wherein said amine component typically is unsubstituted or substituted with at least one of a halogen, a hydroxyl, an amino group, a sulfonyl group, a sulphonamide group, a thiol, a C1-6 alkyl, a C1-C6 alkoxy, a C1-C6 ether, a C1-C6 thioether, a C1-C6 sulfone, a C1-C6 sulfoxide, a C1-C6 primary amide, a C1-C6 secondary amide, a halo C1-C6 alkyl, a carboxyl group, a cyano group, a nitro group, a nitroso group, —OC(0)NR′R′, —N(R′)C(0)NR′R, —N(R′)C(0)0-C1-C6 alkyl, C3-C6 cycloalkyl, C3-C6 heterocyclyl, C2-C5 heteroaryl and C6-C10 aryl; wherein each R′ is independently selected, from the group consisting of hydrogen and C1-C6 alkyl; and
(iv) a second amine component typically comprising 3 to 20 atoms,
   wherein said amine component typically is unsubstituted or substituted with at least one of a halogen, a hydroxyl, an amino group, a sulfonyl group, a sulphonamide group, a thiol, a C1-C6 alkyl, a C1-C6 alkoxy, a C1-C6 ether, a C1-C6 thioether, a C1-C6 sulfone, a C1-C6 sulfoxide, a C1-C6 primary amide, a C1-C6 secondary amide, a halo C1-C6 alkyl, a carboxyl group, a cyano group, a nitro group, a nitroso group, —OC(0)NR′R′, —N(R′)C(0)NR′R, —N(R′)C(0)0-C1-C6 alkyl, C3-C6 cycloalkyl, C3-C6 heterocyclyl, C2-C5 heteroaryl and C6-C10 aryl; wherein each R′ is independently selected, from the group consisting of hydrogen and C1-C6 alkyl.

In one embodiment, the polymer is formed using a oligomer combination approach, in which the diacrylate and first amine components are reacted together to form a first oligomer, the first oligomer and second amine component are reacted together to form a second oligomer, and the second oligomer and four branching monomer are reacted together to form the hyperbranched polymer of the invention. This oligomer combination approach is described in detail in Zeng et al (Nano. Lett. 2019 19, 381-391). In another embodiment, the four-branching monomer, diacrylate component, and first amine are reacted together in a Michael Addition reaction to form a first polymer, and the first polymer and second amine component (endcapping amine) are reacted together in a Michael Addition reaction to form the hyperbranched polymer of the invention.

“Four branching monomer” refers to a component having four reaction sites that can react with acrylate or amine groups. Examples of four-branching monomers include diamine and tetraacrylate components, examples of which are provided above. The scaffold may also be a 4-arm PEG component, a pentaerythritol group, a tetraglycidyl group, or a tetra-substituted silane group. The reactive group may be any acrylamide component (including maleimide), a N-hydroxysuccinimidyl (NHS) component, a thiol component, and an epoxy component. The following are specific examples of four-branching monomers that may be employed in the process and products of the invention:

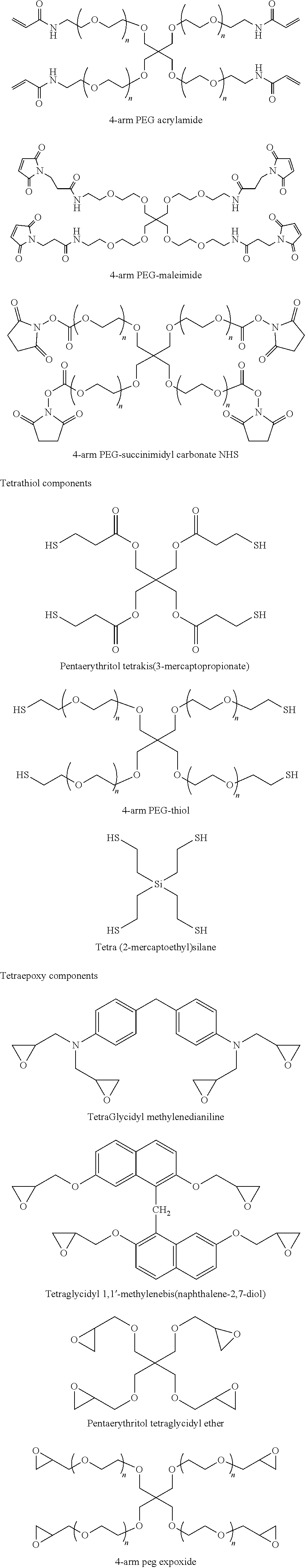

“Linker” means any linker group, including an aryl or alkyl group. Preferred linkers include O, NH, CH₂, alkyl, lower alkyl, alkoxy, lower alkoxy, O-alkyl, CH₂O, CH₂NH, and CH₂NHCOCH₂, CO, COO.

“Diamine component” refers to a moiety having two functional NH2 groups connected by a a linker. “Tetraacrylate” refers to a moiety having four functional acrylate groups.

“Lower alkyl” means an alkyl group, as defined below, but having from one to ten carbons, more preferable from one to six carbon atoms (eg. “C—C—alkyl”) in its backbone structure.

“Alkyl” refers to a group containing from 1 to 8 carbon atoms and may be straight chained or branched. An alkyl group is an optionally substituted straight, branched or cyclic saturated hydrocarbon group. When substituted, alkyl groups may be substituted with up to four substituent groups, at any available point of attachment. When the alkyl group is said to be substituted with an alkyl group, this is used interchangeably with “branched alkyl group”. Exemplary unsubstituted such groups include methyl, ethyl, propyl, isopropyl, a-butyl, isobutyl, pentyl, hexyl, isohexyl, 4,4-dimethyl pentyl, octyl, 2,2,4-trimethylpentyl, nonyl, decyl, undecyl, dodecyl, and the like. Exemplary substituents may include but are not limited to one or more of the following groups: halo (such as F, Cl, Br, I), Haloalkyl (such as CC13 or CF3), alkoxy, alkylthio, hydroxyl, carboxy (—COOH), alkyloxycarbonyl (—C(O)R), alkylcarbonyloxy (—OCOR), amino (—NH2), carbamoyl (—NHCOOR-or-OCONHR), urea (—NHCONHR—) or thiol (—SH). Alkyl groups as defined may also comprise one or more carbon double bonds or one or more carbon to carbon triple bonds.

“Lower alkoxy” refers to O-alkyl groups, wherein alkyl is as defined hereinabove. The alkoxy group is bonded to the core compound through the oxygen bridge. The alkoxy group may be straight-chained or branched; although the straight-chain is preferred. Examples include methoxy, ethyloxy, propoxy, butyloxy, t-butyloxy, i-propoxy, and the like. Preferred alkoxy groups contain 1-4 carbon atoms, especially preferred alkoxy groups contain 1-3 carbon atoms. The most preferred alkoxy group is methoxy. “Halogen” means the non-metal elements of Group 17 of the periodic table, namely bromine, chlorine, fluorine, iodine and astatine.

The terms “alkyl”, “cycloalkyl”, “heterocycloalkyl”, “cycloalkylalkyl”, “aryl”, “acyl”, “aromatic polycycle”, “heteroaryl”, “arylalkyl”, “heteroarylalkyl”, “amino acyl”, “non-aromatic polycycle”, “mixed aryl and non-aryl polycycle”, “polyheteroaryl”, “non-aromatic polyheterocyclic”, “mixed aryl and non-aryl polyheterocycles”, “amino”, and “sulphonyl” are defined in U.S. Pat. No. 6,552,065, Column 4, line 52 to Column 7, line 39.

“Halogen” means the non-metal elements of Group 17 of the periodic table, namely bromine, chlorine, fluorine, iodine and astatine.

“Nanoparticulate composition” refers to a composition is the nano-size range. In one embodiment, the particulate composition has a particle size of less than 2 μm, 1.5 μm, 1000 nm, for example 20-900 nm, 50-800 nm, 50-700 nm, 50-600 nm, 50-500 nm, 50-400 nm, 50-300 nm, 100-300 nm, 150-250 nm, or about 200 nm.

“Gene therapy/editing”: The present invention may be used to edit a portion of the genome of a cell or replace a portion of the genome of a cell with an exogenous DNA insert in an orientation-specific manner.

Thus, the invention may be used to edit or replace a defective portion of a disease-causing gene (i.e. for gene repair), or to insertionally inactivate (i.e. silence) a gene the expression of which is associated with a disease, or to edit or modify a gene for example to delete disease causing mutations or modify or add in residues required for normal functioning of a gene.

Thus, the invention finds application in gene therapy, as herein defined.

Gene therapy according to the invention may target all of the cells in an organism, or may be targeted to a subset of cells (e.g. to selected organs, tissues or cells).

Gene therapy according to the invention may target somatic cells specifically.

Gene therapy according to the invention may exclude the targeting of germ line cells. It may exclude the targeting of totipotent cells. It may exclude the targeting of human embryos.

In cases where gene therapy according to the invention is applied to selected organs, tissues or cells, the method may be applied ex vivo to isolated organs, tissues or cells (e.g. to blood, blood cells, immune cells, bone marrow cells, skin cells, nervous tissue, muscle etc.).

Gene therapy finds application in the treatment of any genetically inherited disorder, particularly those arising from single gene mutations. Thus, gene therapy finds particular application in the treatment of lysosomal storage diseases, muscular dystrophies, cystic fibrosis, Marfan syndrome, sickle cell anaemia, dwarfism, phenylketonuria, neurofibromatosis, Huntington's disease, osteogenesis imperfecta, thalassemia and hemochromatosis.

Other diseases which may be suitable for gene therapy according to the invention include diseases and disorders of: blood, coagulation, heterogenous skin disease, cell proliferation and dysregulation, neoplasia (including cancer), inflammatory processes, immune system (including autoimmune diseases), metabolism, liver, kidney, musculoskeletal, neurological, neuronal and ocular tissues.

Exemplary skin diseases include recessive dystrophic epidermolysis bullosa (RDEB), a rare heterogenous skin disease caused by biallelic loss-of-function mutations in the COL7A 1 gene. Additional skin diseases may include: alternative EB subtypes such as

Simplex EB and Junctional EB, Epidermolytic Palmoplantar Keratoderma, Hailey-Hailey's disease, Darier's disease and Localized Autosomal Recessive Hypotrichosis.

Exemplary blood and coagulation diseases and disorders include: anaemia, bare lymphocyte syndrome, bleeding disorders, deficiencies of factor H, factor H-like 1, factor V, factor VIII, factor VII, factor X, factor XI, factor XII, factor XIIIA, factor XIIIB, Fanconi anaemia, haemophagocytic lymphohistiocytosis, haemophilia A, haemophilia B, haemorrhagic disorder, leukocyte deficiency, sickle cell anaemia and thalassemia.

Examples of immune related diseases and disorders include: AIDS; autoimmune lymphoproliferative syndrome; combined immunodeficiency; HIV-1; HIV susceptibility or infection; immunodeficiency and severe combined immunodeficiency (SCIDs). Autoimmune diseases which can be treated according to the invention include Grave's disease, rheumatoid arthritis, Hashimoto's thyroiditis, vitiligo, type I (early onset) diabetes, pernicious anaemia, multiple sclerosis, glomerulonephritis, systemic lupus E (SLE, lupus) and Sjogren syndrome. Other autoimmune diseases include scleroderma, psoriasis, ankylosing spondilitis, myasthenia gravis, pemphigus, polymyositis, dermomyositis, uveitis, Guillain-Barre syndrome, Crohn's disease and ulcerative colitis (frequently referred to collectively as inflammatory bowel disease (IBD)).

Other exemplary diseases include: amyloid neuropathy; amyloidosis; cystic fibrosis; lysosomal storage diseases; hepatic adenoma; hepatic failure; neurologic disorders; hepatic lipase deficiency; hepatoblastoma, cancer or carcinoma; medullary cystic kidney disease; phenylketonuria; polycystic kidney; or hepatic disease.

Exemplary musculoskeletal diseases and disorders include: muscular dystrophy (e.g. Duchenne and Becker muscular dystrophies), osteoporosis and muscular atrophy.

Exemplary neurological and neuronal diseases and disorders include: ALS, Alzheimer's disease; autism; fragile X syndrome, Huntington's disease, Parkinson's disease,

Schizophrenia, secretase related disorders, trinucleotide repeat disorders, Kennedy's disease, Friedrich's ataxia, Machado-Joseph's disease, spinocerebellar ataxia, myotonic dystrophy and dentatorubral pallidoluysian atrophy (DRPLA).

Exemplary ocular diseases include: age related macular degeneration, corneal clouding and dystrophy, cornea plana congenital, glaucoma, Leber's congenital amaurosis and macular dystrophy.

Gene therapy according to the invention finds particular application in the treatment of lysosomal storage disorders. Listed below are exemplary lysosomal storage disorders and the corresponding defective enzymes:

| | |
|---|---|
| Pompe disease: | Acid alpha-glucosidase |
| Gaucher disease: | Acid beta-glucosidase or |
| | glucocerebrosidase |
| Fabry disease: | alpha-Galactosidase A |
| GMI-gangliosidosis: | Acid beta-galactosidase |
| Tay-Sachs disease: | beta-Hexosaminidase A |
| Sandhoff disease: | beta-Hexosaminidase B |
| Niemann-Pick disease: | Acid sphingomyelinase |
| Krabbe disease: | Galactocerebrosidase |
| Farber disease: | Acid ceramidase |
| Metachromatic leukodystrophy: | Arylsulfatase A |
| Hurler-Scheie disease: | alpha-L-Iduronidase |
| Hunter disease: | Iduronate-2-sulfatase |
| Sanfilippo disease A: | Heparan N-sulfatase |
| Sanfilippo disease B: | alpha-N-Acetylglucosaminidase |
| Sanfilippo disease C: | Acetyl-CoA: alpha-glucosaminide |
| | N-acetyltransferase |
| Sanfilippo disease D: | N-Acetylglucosamine-6-sulfate |
| | sulfatase |
| Morquio disease A: | N-Acetylgalactosamine-6-sulfate |
| | sulfatase |
| Morquio disease B: | Acid beta-galactosidase |
| Maroteaux-Lamy disease: | Arylsulfatase B |
| Sly disease: | beta-Glucuronidase |
| alpha-Mannosidosis: | Acid alpha-mannosidase |
| beta-Mannosidosis: | Acid beta-mannosidase |
| Fucosidosis: | Acid alpha-L-fucosidase |
| Sialidosis: | Sialidase |
| Schindler-Kanzaki disease: | alpha-N-acetylgalactosaminidase |

Gene therapy according to the invention also finds particular application in the treatment of proteostatic diseases including both aggregative and misfolding proteostatic diseases, for example prion diseases, various amyloidoses and neurodegenerative disorders (e.g. Parkinson's disease, Alzheimer's disease and Huntington's disease), certain forms of diabetes, emphysema, cancer and cystic fibrosis.

Gene therapy according to the invention finds particular application in the treatment of cystic fibrosis. Cystic fibrosis occurs when there is a mutation in the CFTR gene leading to reduced ion channel activity (via increased clearance of the misfolded CFTR proteins).

Gene therapy according to the invention finds particular application in the treatment of expanded CAG repeat diseases. These diseases stem from the expansion of CAG repeats in particular genes with the encoded proteins having corresponding polyglutamine tracts which lead to aggregation and accumulation in the nuclei and cytoplasm of neurons. Aggregated amino-terminal fragments of mutant huntingtin are toxic to neuronal cells and are thought to mediate neurodegeneration. Examples include Huntington's disease (HD), which is characterized by selective neuronal cell death primarily in the cortex and striatum. CAG expansions have also been found in at least seven other inherited neurodegenerative disorders, including for example spinal and bulbar muscular atrophy (SBMA), Kennedy's disease, some forms of amyotrophic lateral sclerosis (ALS), dentatorubral pallidoluysian atrophy (DRPLA) and spinocerebellar ataxia (SCA) types 1, 2, 3, 6 and 7.

Gene therapy according to the invention finds particular application in the treatment of any neoplasia, including proliferative disorders, benign, pre-cancerous and malignant neoplasia, hyperplasia, metaplasia and dysplasia. The invention therefore finds application in the treatment of proliferative disorders which include, but are not limited to cancer, cancer metastasis, smooth muscle cell proliferation, systemic sclerosis, cirrhosis of the liver, adult respiratory distress syndrome, idiopathic cardiomyopathy, lupus erythematosus, retinopathy (e.g. diabetic retinopathy), cardiac hyperplasia, benign prostatic hyperplasia, ovarian cysts, pulmonary fibrosis, endometriosis, fibromatosis, hematomas, lymphangiomatosis, sarcoidosis and desmoid tumours. Neoplasia involving smooth muscle cell proliferation include hyperproliferation of cells in the vasculature (e.g. intimal smooth muscle cell hyperplasia, restenosis and vascular occlusion, including in particular stenosis following biologically- or mechanically-mediated vascular injury, such as angioplasty). Moreover, intimal smooth muscle cell hyperplasia can include hyperplasia in smooth muscle other than the vasculature (e.g. blockage of the bile duct, bronchial airways and in the kidneys of patients with renal interstitial fibrosis). Non-cancerous proliferative disorders also include hyperproliferation of cells in the skin such as psoriasis and its varied clinical forms, Reiter's syndrome, pityriasis rubra pilaris and hyperproliferative variants of disorders of keratinization (including actinic keratosis, senile keratosis and scleroderma). Particularly preferred is the treatment of malignant neoplasia (cancer).

### Administration

The composition of the invention may be adapted for topical, oral, rectal, parenteral, intramuscular, intraperitoneal, intra-arterial, intrabronchial, subcutaneous, subdermal, intradermal, intravenous, nasal, vaginal, buccal, ocular or sublingual routes of administration. For oral administration, particular use is made of compressed tablets, pills, tablets, drops, and capsules. Preferably, these compositions contain from 0.01 to 250 mg and more preferably from 0.1-10 mg, of active ingredient per dose. Other forms of administration comprise solutions or emulsions which may be injected intravenously, intra-arterial, subcutaneously, intradermally, intraperitoneally or intramuscularly, and which are prepared from sterile or sterilisable solutions. The pharmaceutical compositions of the present invention may also be in form of suspensions, emulsions, lotions, ointments, creams, gels, sprays, nebulizers, solutions or dusting powders. The composition of the invention may be formulated for topical delivery. Topical delivery generally means delivery to the skin, but can also mean delivery to a body lumen lined with epithelial cells, for example the lungs or airways, the gastrointestinal tract, the buccal cavity. In particular, formulations for topical delivery are described in Topical drug delivery formulations edited by David Osborne and Antonio Aman, Taylor & Francis, the complete contents of which are incorporated herein by reference. Compositions or formulations for delivery to the airways are described in O'Riordan et al (Respir Care, 2002, Nov. 47), EP2050437, WO2005023290, US2010098660, and US20070053845. Composition and formulations for delivering active agents to the ileum, especially the proximal ileum, include microparticles and microencapsulates where the active agent is encapsulated within a protecting matrix formed of polymer or dairy protein that is acid resistant but prone to dissolution in the more alkaline environment of the ileum. Examples of such delivery systems are described in EP1072600.2 and EP13171757.1. An alternative means of transdermal administration is by use of a skin patch. For example, the active ingredient can be incorporated into a cream consisting of an aqueous emulsion of polyethylene glycols or liquid paraffin or into a hydrogel. The active ingredient can also be incorporated, at a concentration of between 1 and 10% by weight, into an ointment consisting of a white wax or white soft paraffin base together with such stabilisers and preservatives as may be required. Injectable forms may contain between 10-1000 mg, preferably between 10-250 mg, of active ingredient per dose. Compositions may be formulated in unit dosage form, i.e., in the form of discrete portions containing a unit dose, or a multiple or sub-unit of a unit dose.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention. Depending upon the need, the agent may be administered at a dose of from 0.01 to 50 mg/kg body weight, such as from 0.1 to 10 mg/kg, more preferably from 0.1 to 1 mg/kg body weight.

The term “pharmaceutically acceptable excipient” refers to a diluent, adjuvant, excipient, or vehicle with which the polyplex is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents, or skin penetration enhancers. These compositions can take the form of solutions, suspensions, emulsion, tablets, pills, capsules, powders, sustained-release formulations and the like.

### Exemplification

The invention will now be described with reference to specific examples. These are merely exemplary and for illustrative purposes only: they are not intended to be limiting in any way to the scope of the monopoly claimed or to the invention described. These examples constitute the best mode currently contemplated for practicing the invention.

### Synthesis of Cationic Polymer (Y Polymer (4-Branching Diamine))

An embodiment of a 4-branching diamine hyperbranched cationic polymer was prepared according to Scheme 1 below, using BDA, EDA (or HMDA), S5 and DA (or DATA) monomers.

To synthesize the cationic Y polymer the monomers: BDA, EDA (or HMDA), S5, and DA (or DATA) were used. The Y polymer was formed through an “A2+B4+C2” Michael addition strategy using the copolymerization of commercially available monomers. Each selected monomer within the reaction system plays a pivotal role in the final Y4 polymer. Diamine monomer (B4) was employed as the branching unit to generate the highly branched polymer through combination with linear diacrylate monomer (A2). Further post synthesis modification involved end capping polymer terminal groups with additional (Amine monomer, C2) to remove any unreacted vinyl groups. Monomers were added into a round bottomed flask with a magnetic stirring bar. The flask was placed partially submerged in an oil bath and polymerization reactions were carried out at 90° C. Gel permeation chromatography (GPC) was used to track the progression of the polymer synthesis reaction by measuring molecular weight, conversion and PDI. Upon polymer molecular weight (Mw) approaching 10-20 kDa the reaction was stopped by removing from heat and diluted with DMSO. Y polymers chain termination was achieved by reacting the polymer solution with the amine end capping agent for 48 hrs at room temperature. Post end-capping, Y polymers were purified by precipitating in excess diethyl ether twice so as to remove any remaining residual monomers, unreacted end capping agents and small oligomers. To achieve the final product, Y polymers were dried in a vacuum oven for 48 hrs to remove remaining solvents.

### Synthesis of Cationic Polymer (P Polymers (4-Branching Tetraacrylate, P1, P2 and P3)

An embodiment of a 4-branching tetraacrylate hyperbranched cationic polymer was prepared according to Scheme 2 below, using BDA, PTTA (or DTTA), S5 and DA (or DATA) monomers. BDA, PTTA (or DTTA), and S5 were mixed into a flask with DMSO as the solvent. The reaction was performed at 90° C. until the target Mw was achieved. The reaction was stopped by removing the reaction flask from heat and cooled with ice. End-capping monomer DA (or DATA) was added into the flask with DMSO to react with the acrylate residual for 48 hrs at room temperature. Afterwards, the reaction mixture was precipitated into excess amount of diethyl ether twice to remove the monomers and oligomers. The P1 and P2 polymers (BDA+PTTA+S5+DA) were achieved by drying in a vacuum oven with 7 kDa and 10 kDa Mw, respectively. P3 polymers (BDA+PTTA+S5+DATA) was achieved by the same procedure with 10 kDa Mw.

Synthesis of Cationic Polymer (HPAE polymer—3-Branching Triacrylate)

4-amino-1-butanol (S4), trimethylolpropane triacrylate (TMPTA) and bisphenol A ethoxylate diacrylate (BE) were polymerized via a one-pot “A2+B3+C2′ type Michael addition. Then, functional 3-morpholinopropylamine (MPA) was introduced by end capping to further enhance the property and functionalities of HPAEs as gene vectors (WO2016/020474).

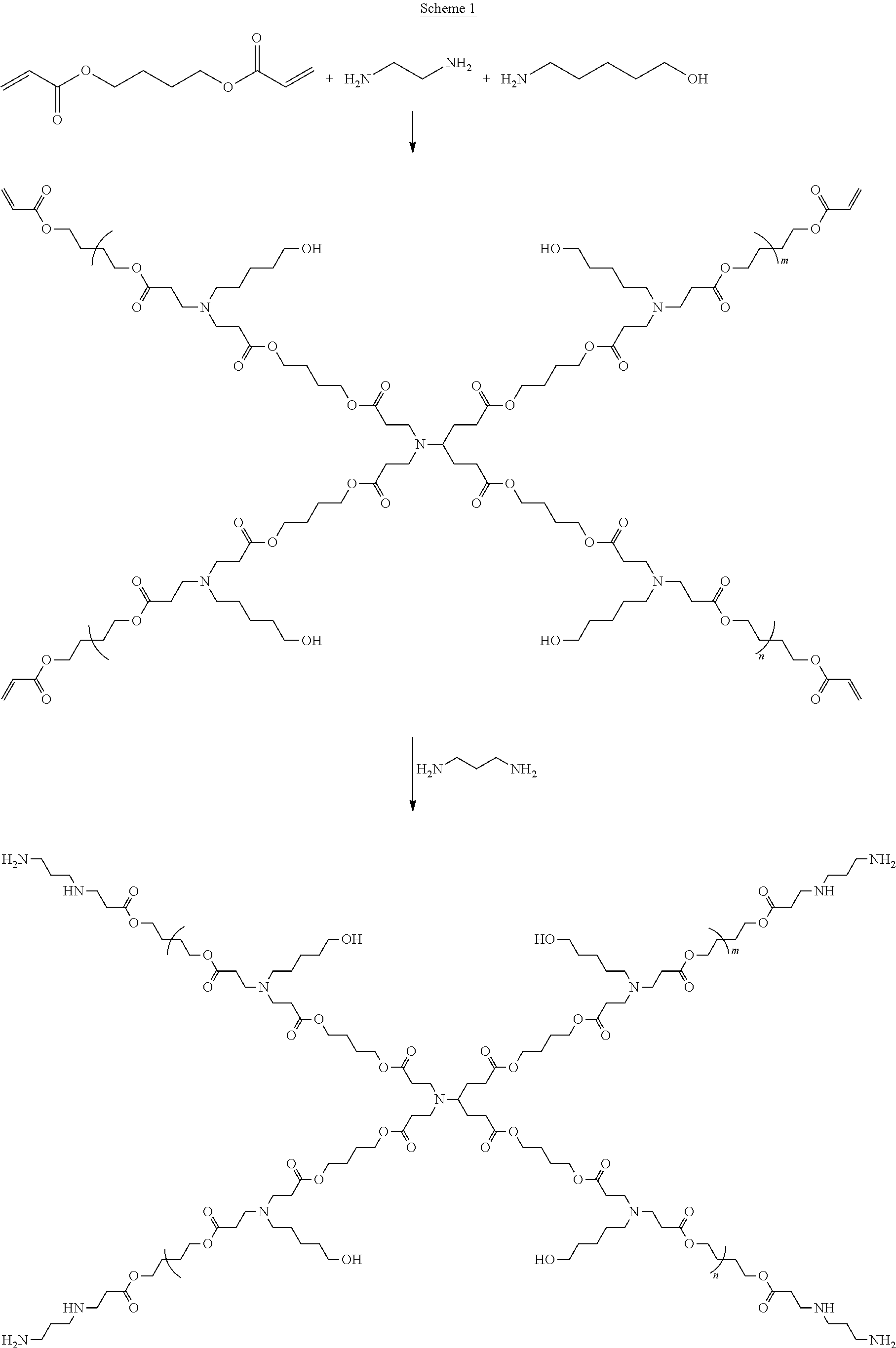

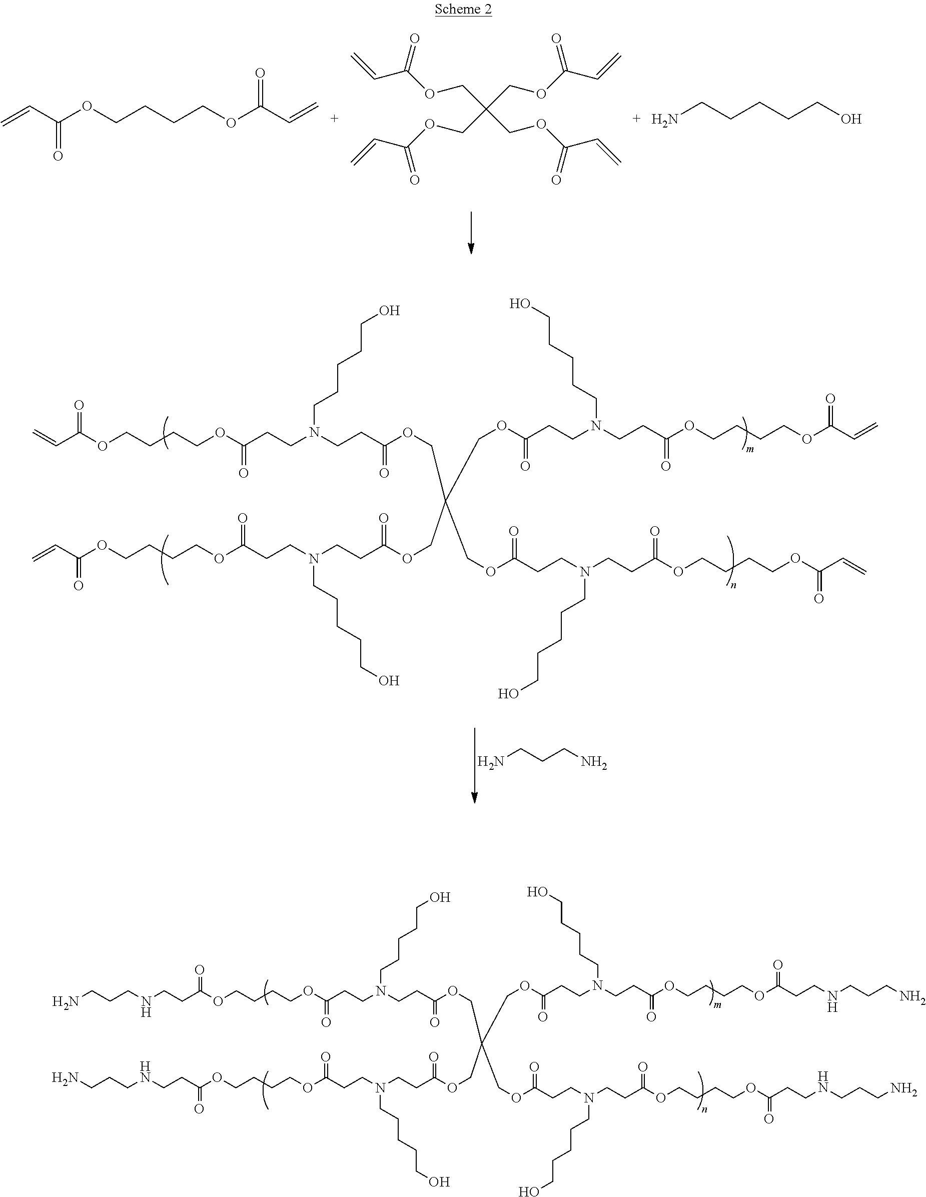

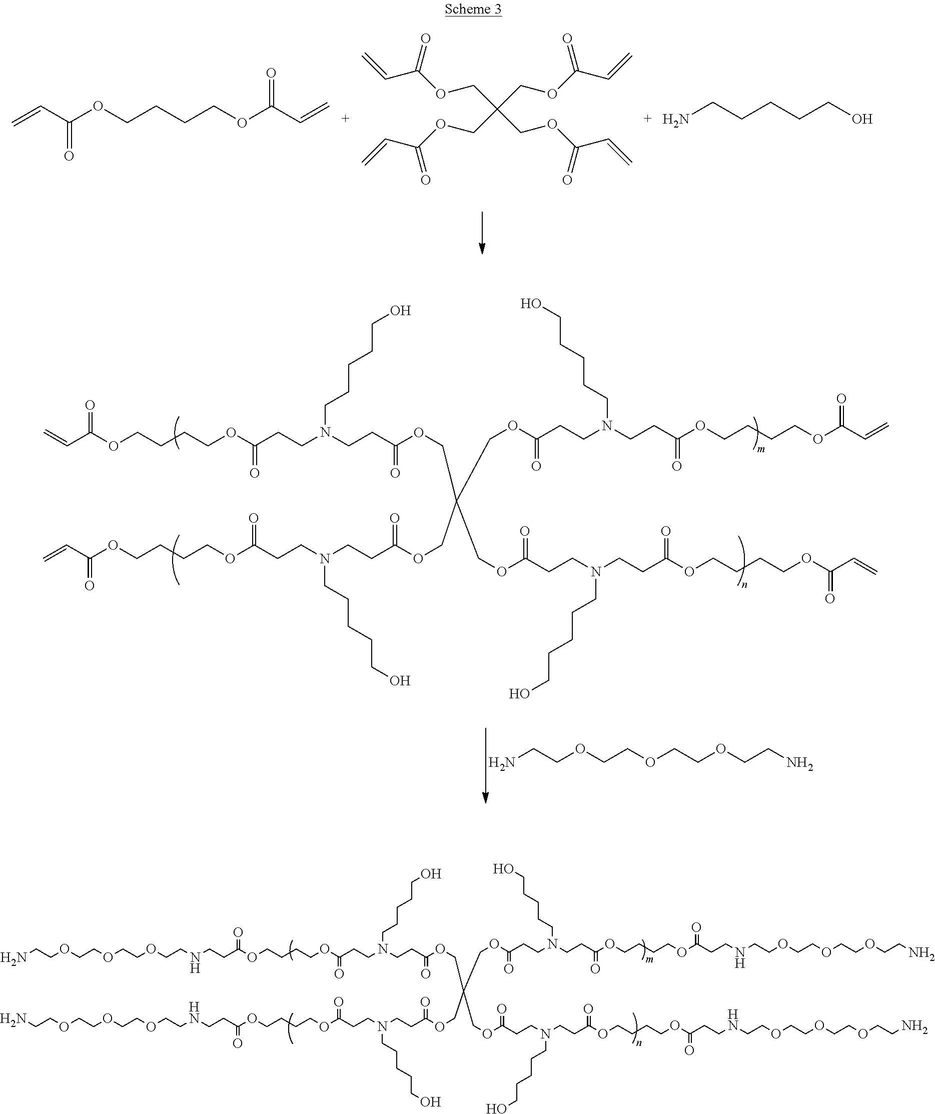

### Synthesis of Nanoparticulate Composition of the Invention (Ribopolyplex)

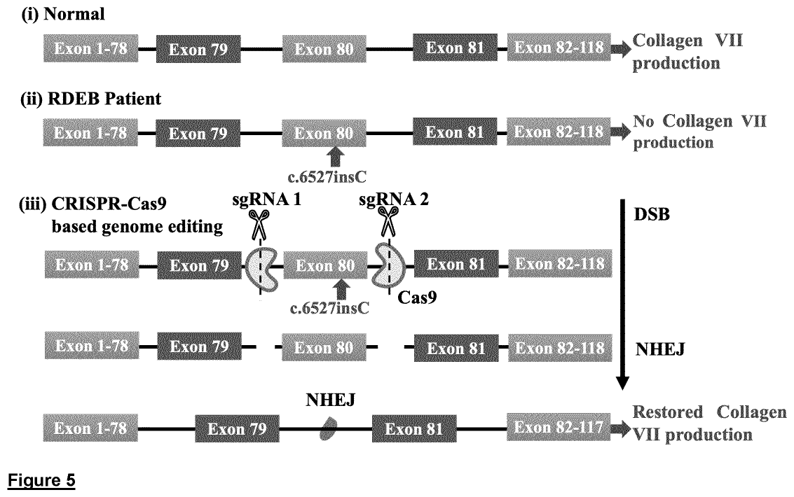

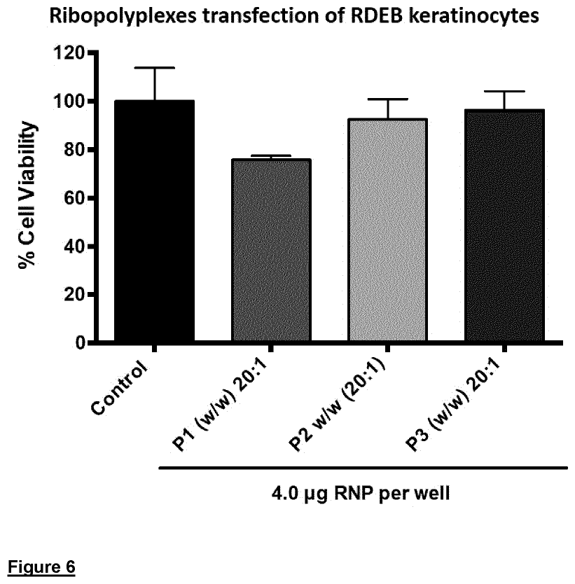

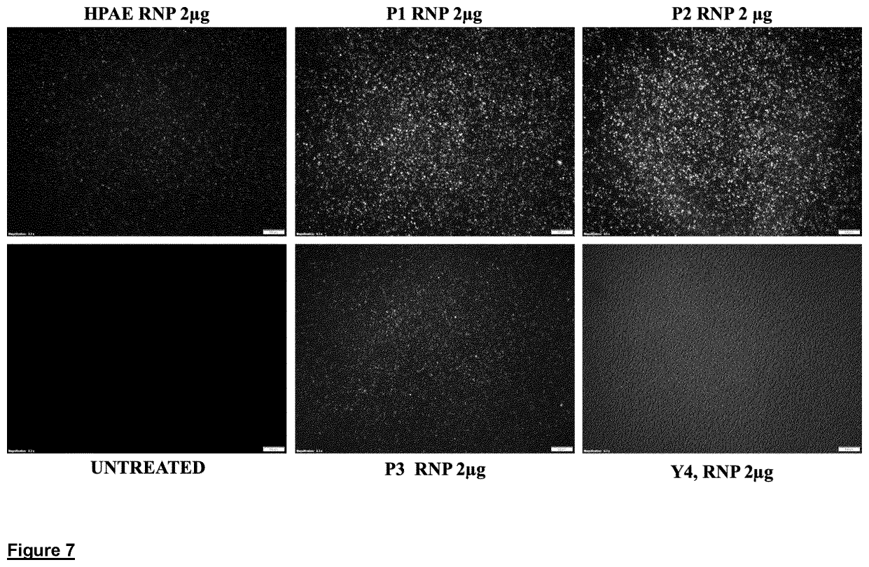

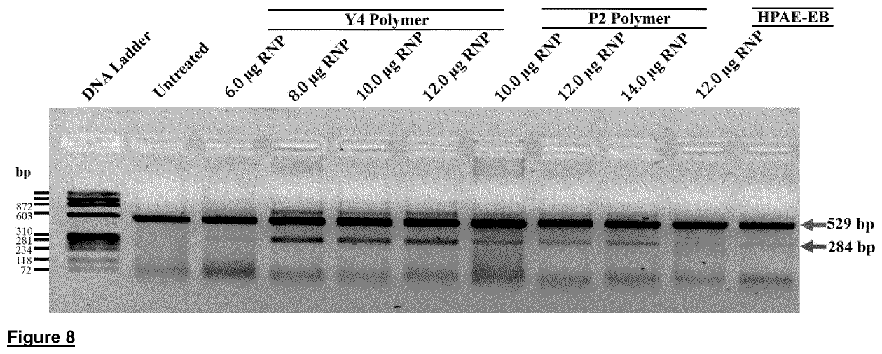

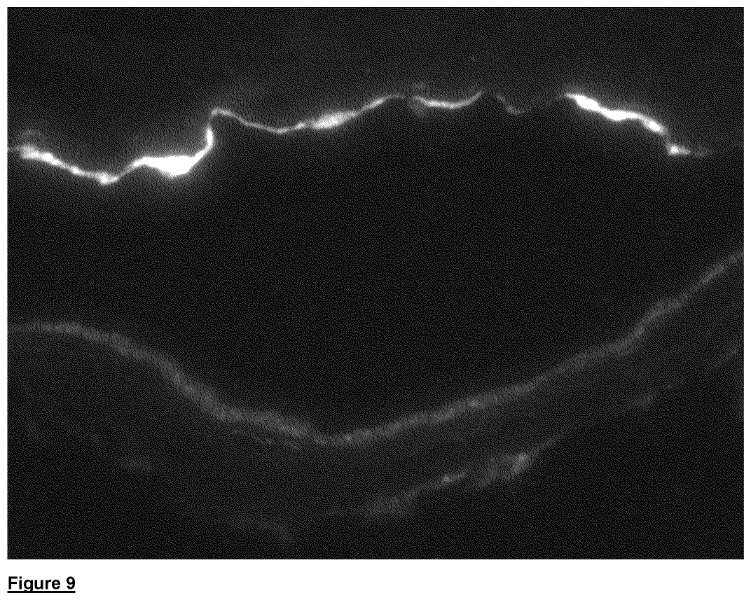

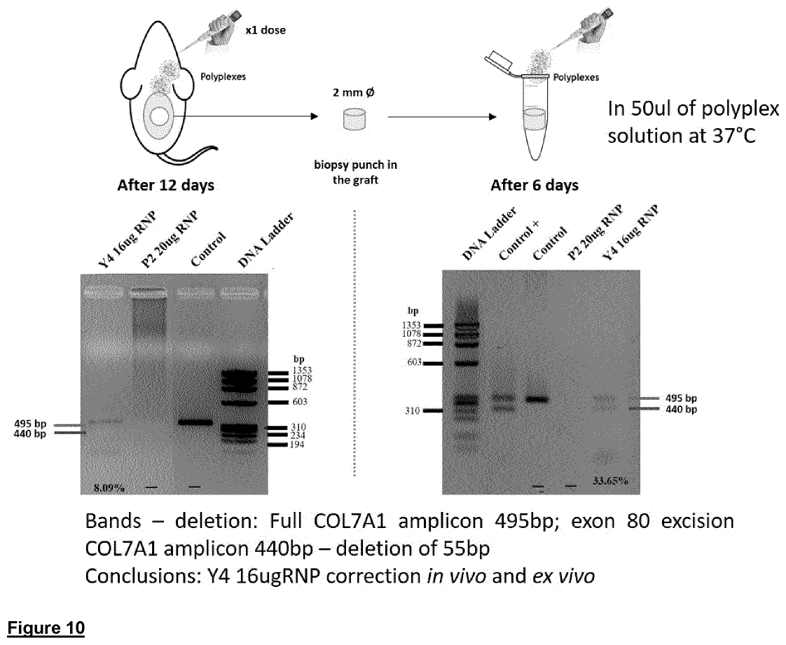

Ribopolyplexes are formed by first preparing aqueous polymer and ribonucleoprotein complex solutions in a suitable solvent, for example 25 mM sodium acetate, 1:1 v/v ratio of dissolved ribonucleoprotein and dissolved polymer are mixed together, polymer- ribonucleoprotein complex solution is incubated at room temperature for 10 min to allow ribopolyplex formation prior to use. The aqueous ribopolyplex solution should be mixed vigorously to ensure solution homogeneity. The ribonucleoprotein complex was complexed with different cationic polymers (P1, P2 and P3 described above). For this specific application, the ribonucleoprotein complex was developed for treatment of Recessive Dystrophic subtype of Epidermolysis Bullosa (RDEB). The strategy followed for RDEB using the ribopolyplexes is the collagen VII exon 80 excision (FIG. 5), using a RNP complex formed by Cas9 and 2 single guide RNA's complexed with a fluorescently labelled red tracrRNA. Collagen VII exon 80 contains a high number of mutations for RDEB, like the most common one (c.6527insC), that produces a stop codon. Excising the exon, the collagen VII obtained is completely functional, ameliorating the symptoms with only a 30% production of corrected protein. Both crRNAs and tracrRNA are diluted to 100 μM with nuclease free duplex buffer with HiFi Cas9 nuclease used at stock concentration of 62 μM as per manufacturers guidelines. RNP complexes are prepared such that sgRNA(crRNA+tracrRNA): Cas9 molar ratio is 1-9:1. Master mixes are heated for 5 min at 95° C. in a thermocycler to anneal crRNA and tracrRNA. Following this, they are removed from heat and allowed to cool to room temperature on bench top. To each master mix HiFi Cas9 nuclease is added and allowed to complex for 15 min at room temperature, protected from light. Polymer:RNP polyplexes (ribopolyplexes) are prepared for transfection in a similar manner to that of plasmid DNA based transfections. Polymer is diluted in 25 mM sodium acetate buffer to desired concentrations as previously described. Equal amounts of each RNP master mix 1 and 2 are used for every transfection. To calculate the w/w ratio for the polymer, the entire weight of the RNP complex is used. To form complexes, polymer solutions are mixed with RNP solutions at a 1:1 v/v ratio. After mixing together by pipetting up and down, complexes are incubated at room temperature for 15 min to allow polymer-RNP interactions. Once incubation is completed, ribopolyplex solutions are ready to be diluted in appropriate cell culture media and added to cells. 4 hrs after transfection, media is changed and replaced with fresh culture media. An ATTO 550 nm fluorophore on the tracrRNA is used an indicator of transfection efficiency.

### Transfected Cell Viability

Evaluation of cell cytotoxicity induced by different polyplex conditions has been assessed using the alamarBlue™ assay, which provided a quantitative measurement of cell proliferation and metabolic health. Cell viability has been assessed 48-72 hrs post transfection experiments in cells. Culture media is removed from cells in a well plate and cells are washed with (hanks balanced salt solution) HBSS per well. Following this, 100 μl of alamarBlue™ working solution (10% alamarBlue™ in HBSS) is added to each well and allowed to incubate under normal cell culture conditions for 2 hrs protected from light. After incubation, the alamarBlue™ solution is transferred to a fresh flat bottomed 96 well plate and absorbance at 570 nm and 600 nm is recorded on a SpectraMax M3 multi-plate reader. Wells containing alamarBlue™ reagent only are subtracted from each sample as a background reading. Untreated cells are used to normalize fluorescence values and plotted as 100% viable.

### Cell Transfection Efficiency

Cells are seeded 24 hr-48 hrs prior to transfections to allow attachment to well plates and flasks. Cells are seeded at optimized cell densities. On the day of transfection, polymer-DNA complexes are prepared and after complexation, are mixed with the appropriate cell media such that the final polyplex solution is no more than 20% of the overall media volume. Cell media containing polymer-DNA complexes are added to cells and after 4 hrs is removed and replaced with fresh media to remove complexes.

### In Vivo Application of the Ribopolyplexes Using Different Polymers in RDEB Human Graft Model

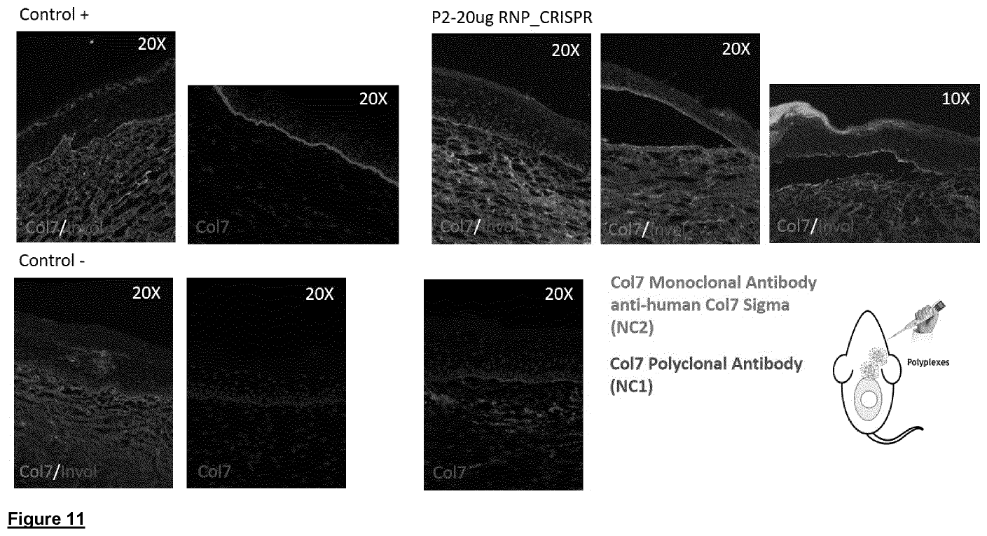

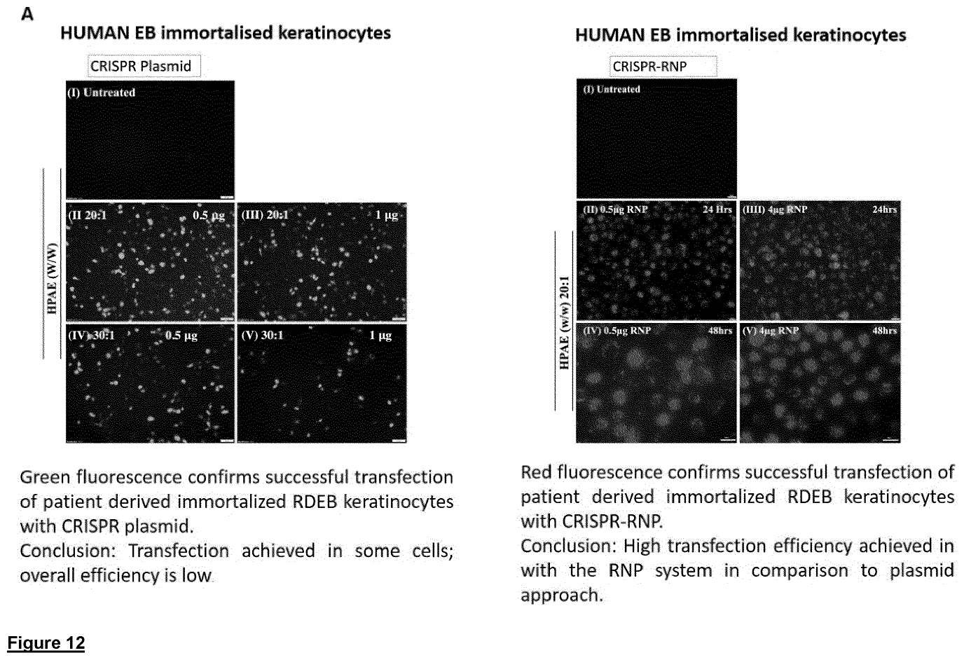

The efficiency in vivo of the ribopolyplexes can be evaluated in an established skin humanized mouse model system based on bioengineered human skin-engrafted immunodeficient mice, where human fibroblasts and keratinocytes isolated from a skin biopsy are expanded in vitro to produce RDEB human bioengineered skin. The tissue bioengineered skin equivalent is then grafted into an athymic mouse. The skin-humanized mouse model based on the stable engraftment of this setting represents a useful pre-clinical platform to the model pathophysiological process and to test innovative therapeutic protocols. The ribopolyplex suspension is topically and/or intradermally applied to the RDEB graft after demarcation of the treatment surface with petroleum jelly or in a simulated wound within the graft. After determinate period of time, the graft can be tested for structural stability by mechanically pulling the graft, also 2 mm punch biopsy can be taken to evaluate the correction efficiency. At the end of the assessment, the graft tissue is evaluated for corrected bands detection by PCR (FIG. 10), collagen VII immunofluorescence (FIG. 11), histological evaluation and anchoring fibrils confirmation by transmission electron microscopy (TEM).

### Transfection Efficiency of 3-Branched Polymers Gene Editing Systems: Plasmid V Ribonucleoprotein (RNP)

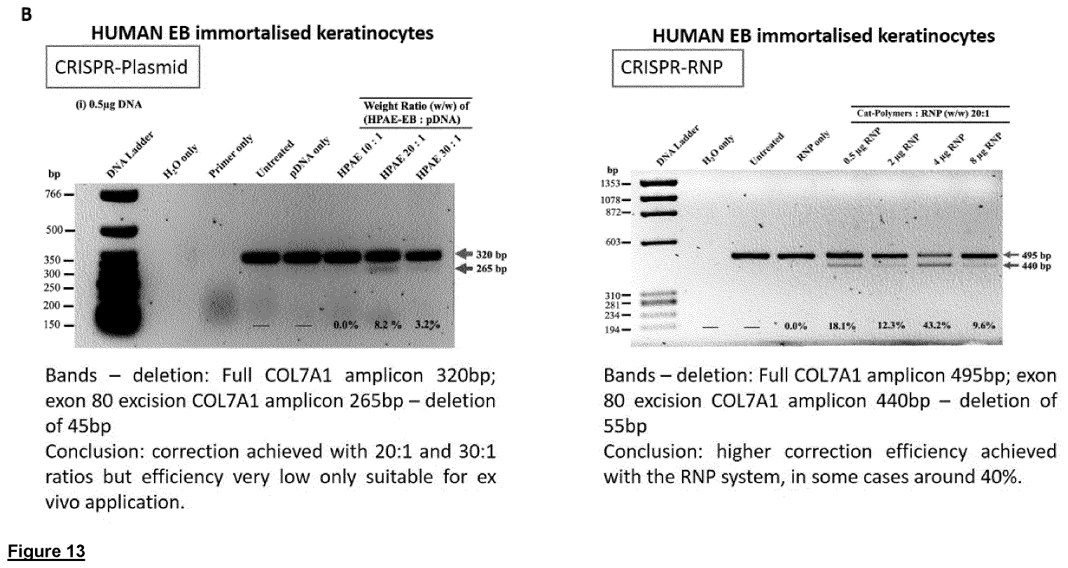

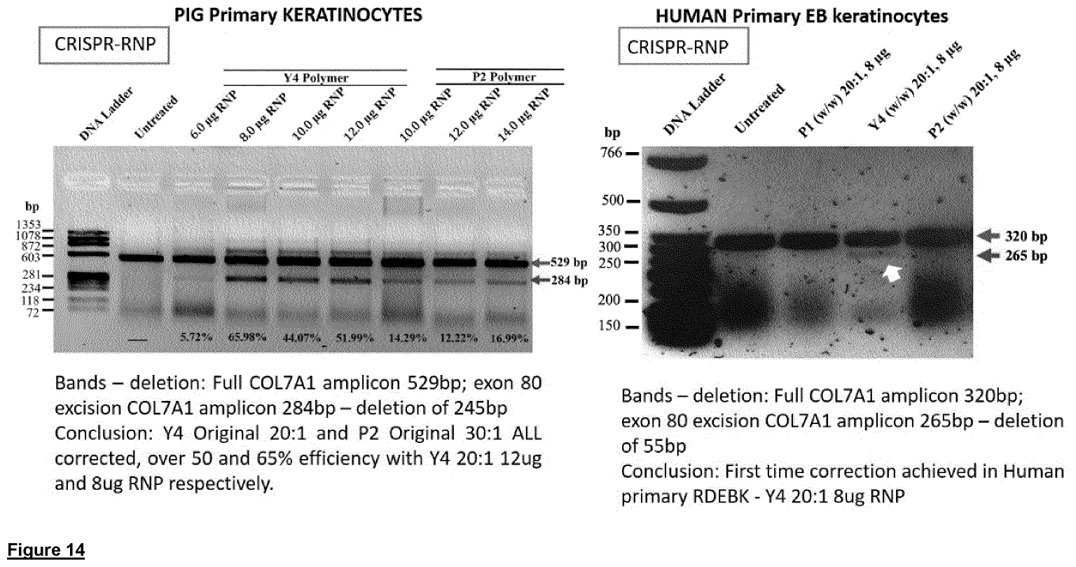

Immortalised RDEB keratinocytes containing a mutation exon 80 were seeded onto well plates. After 24 hrs, transfections with CRISPR-Cas9 plasmid or CRISPR-Cas9-RNP complex were performed, to correct the keratinocyte cells by excising the mutant exon 80 using a dual RNA guide system. Plates were incubated for 4 hours with complexes and afterwards the media was replenished for fresh media. 48 hours after transfection, fluorescence images were taken where the reporter GFP protein (green) from the CRISPR-Cas9 plasmid system and the fluorescent tracrRNA (red) label the cells that have been transfected (FIG. 12). After that cells where trypsinized, DNA was extracted and PCR amplified. The PCR product was run on an agarose gel electrophoresis to confirm successful COL7A1 correction via the presence of a smaller band product representative of DNA lacking exon 80. FIG. 13 displays two different PCR amplicon sizes as a result of differing primer systems used. Using 3 branched polymers with CRISPR DNA plasmid achieved 8.2% correction efficiency however using the CRISPR RNP complex system the efficiency improved up to 43.2% with the same w/w ratio. The fluorescence microscope images along with the PCR results show that the RNP complex achieved higher transfection and correction efficiency.

### Transfection Efficiency of Gene Editing Ribopolyplexes: 3-Branched Polymers v 4-Branched Polymers

Primary keratinocytes from different sources, healthy pig and RDEB human, were transfected with the same CRISPR-Cas9 RNP complex and using the same protocol used to transfect the immortalised RDEB keratinocytes with 3-branched polymers. Of note, 3 branched polymers were used for transfecting in immortalised cells which are well established to be easier to transfect than primary cells. DNA was extracted 48 hours post transfection and PCR amplified; agarose gel electrophoresis shows the correction bands as a result of the exon 80 excision (amplicons with different sizes due to the use of different primers for cell source). Correction efficiencies achieved with Y4 Polymers and CRISPR RNP complex system reached 65.98% in pig primary keratinocytes, significantly higher than that achieved with 3-branched polymers in immortalized cells (FIG. 14 left side). Correction efficiency using Y4 polymers and was achieved even in RDEB human primary keratinocytes, a known difficult to transfect cell (FIG. 14 right side).

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A nanoparticulate composition comprising a gene editing ribonucleoprotein system complexed within a hyperbranched hydrophobic cationic polymer.

2. A nanoparticulate composition according to claim 1, in which the hyperbranched hydrophobic cationic polymer is a 3-branching poly-beta amino ester hyperbranched polymer or a 4-branching poly-beta amino ester hyperbranched polymer.

3. A nanoparticulate composition according to claim 1, in which the hyperbranched hydrophobic cationic polymer is a 4-branching poly-beta amino ester hyperbranched polymer.

4. A nanoparticulate composition according to claim 1, in which the gene editing ribonucleoprotein system is a CRISPR-Cas gene editing system configured to excise a mutation or exon in a gene, replace a mutation in a gene, or produce a knock-down or knock-out of a gene.

5. A nanoparticulate composition according to claim 1, in which the gene editing ribonucleoprotein system is a CRISPR-Cas9 gene editing system configured to excise exon 80 of the COL7A1 gene which codes for the collagen VII protein.

6. A nanoparticulate composition according to claim 1 having an average dimension of 100 nm to 300 nm.

7-28. (canceled)

29. A nanoparticulate composition according to claim 1, in which the hyperbranched hydrophobic cationic polymer is a hyperbranched poly (beta-amino) ester polymer.
